(19) United States
(12) Patent Application Publication
(10) Pub. No.: US 2025/0223616 A1
(43) Pub. Date: Jul. 10, 2025

(54) METHODS FOR PRODUCTION OF CANNABINOIDS

(71) Applicant: BUZZELET DEVELOPMENT AND TECHNOLOGIES LTD., Or Akiva (IL)

(72) Inventor: Aharon EYAL, Jerusalem (IL)

(21) Appl. No.: 18/854,560

(22) PCT Filed: Apr. 10, 2023
(86) PCT No.: PCT/IB2023/053642

§ 371 Date: Oct. 7, 2024

Related U.S. Application Data
(60) Provisional application No. 63/329,894, filed on Apr. 12, 2022.

(51) Int. Cl. C12P 7/42; C12P 5/00

(52) U.S. Cl. CPC C12P 7/42; C12P 5/00

(57) ABSTRACT

Provided is a method for producing at least one cannabinoid, the method comprising providing a cell capable of producing and secreting at least one cannabinoid; providing an organic solvent or an anion exchanger; growing the cell in a growth vessel in an aqueous fermentation medium comprising a carbon source, whereby the cell produces and secretes the at least one cannabinoid; contacting the cannabinoid-comprising fermentation medium with the organic solvent or the anion-exchanger, forming a cannabinoid-comprising organic solvent or a cannabinoid-carrying anion exchanger and a cannabinoid-depleted fermentation medium; and separating the cannabinoid-comprising organic solvent or the cannabinoid-carrying anion exchanger from the cannabinoid-depleted fermentation medium.

### CROSS-REFERENCE TO RELATED APPLICATIONS

The present application gains priority from U.S. Provisional Patent Application Ser. No. 63/329,894 filed Apr. 12, 2022, which is incorporated by reference as if fully set-forth herein.

### Field of the Invention

The present invention relates to the field of cannabinoid production, and more specifically to a method for producing at least one cannabinoid comprising growing cannabinoid-producing cells in an aqueous fermentation medium and contacting the aqueous fermentation medium with an organic solvent or an anion exchanger.

### Background

Cannabinoids are typically provided by growing the Cannabis plant, a method entailing costs such as those related to greenhouse construction, climate control and plant protection. There are also unavoidable differences between products depending on exposure to light and other agricultural parameters. Alternatively, it is known in the art of Cannabis production that cannabinoids can be produced via a dedicated biosynthetic route in isolated Cannabis plant cells, and/or in a host cell, such as a bacterial or yeast cell. However, this alternative has not been commercially adopted, mainly due to limited product concentration and limited growth rate, resulting from the cytotoxic effect of the produced cannabinoid on the plant or host cell.

There is thus an unmet need for an improved method of cannabinoid production, which is devoid of at least some of the disadvantages of the prior art methods.

### SUMMARY OF THE INVENTION

According to an aspect of some embodiments of the present invention, there is provided a method for producing at least one cannabinoid, the method comprising:
(i) providing at least one cell capable of producing at least one cannabinoid and capable of secreting said at least one cannabinoid;
(ii) providing an organic solvent;
(iii) growing said cell in a growth vessel in an aqueous fermentation medium comprising a carbon source, whereby said cell produces and secretes said at least one cannabinoid into said fermentation medium, forming a cannabinoid-comprising fermentation medium;
(iv) contacting said cannabinoid-comprising fermentation medium with said organic solvent, whereby secreted cannabinoid transfers into said organic solvent forming a cannabinoid-comprising organic solvent and a cannabinoid-depleted fermentation medium; and
(v) separating said cannabinoid-containing organic solvent from said fermentation medium, whereby a separated organic solvent comprising said cannabinoid is formed.

According to a further aspect of some embodiments of the present invention, there is provided a method for producing at least one cannabinoid, the method comprising:
(i) providing at least one cell capable of producing at least one cannabinoid and capable of secreting said at least one cannabinoid;
(ii) providing an anion exchanger;
(iii) growing the cell in a growth vessel in an aqueous fermentation medium comprising a carbon source, whereby the cell produces and secretes the at least one cannabinoid into the fermentation medium, forming a cannabinoid-comprising fermentation medium;
(iv) contacting the cannabinoid-comprising fermentation medium with the anion exchanger, whereby secreted cannabinoid binds onto the anion exchanger, forming a cannabinoid-carrying anion exchanger and a cannabinoid-depleted fermentation medium; and
(v) separating the cannabinoid carrying anion exchanger from the cannabinoid-depleted fermentation medium, whereby a separated anion exchanger carrying the cannabinoid is formed.

### BRIEF DESCRIPTION OF THE FIGURES

Some embodiments of the invention are described herein with reference to the accompanying figure. The description, together with the figure, makes apparent to a person having ordinary skill in the art how some embodiments of the invention may be practiced. The figure is for the purpose of illustrative discussion and no attempt is made to show structural details of an embodiment in more detail than is necessary for a fundamental understanding of the invention. For the sake of clarity, some objects depicted in the figure are not to scale.

In the Figure:

FIG. 1 schematically illustrates an exemplary embodiment of a method for the production of a cannabinoid, in accordance with the principles of the present invention.

FIG. 2 schematically illustrates an alternative exemplary embodiment of a method for the production of a cannabinoid, in accordance with the principles of the present invention.

### DETAILED DESCRIPTION OF THE INVENTION

The present invention relates to an improved method for the production of cannabinoids, wherein cannabinoid-producing cells are grown in an aqueous fermentation medium and the aqueous fermentation medium is contacted with an organic solvent or an anion exchanger.

The invention overcomes the problem of the cytotoxic effect of cannabinoids on cannabinoid-producing plants cells and/or on host cells such as bacteria or yeast cells, thereby increasing the cannabinoid yield.

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method for producing at least one cannabinoid, the method comprising:
(i) providing at least one cell capable of producing at least one cannabinoid and capable of secreting the at least one cannabinoid;
(ii) providing an organic solvent;
(iii) growing the cell in a growth vessel in an aqueous fermentation medium comprising a carbon source, whereby the cell produces and secretes the at least one cannabinoid into the fermentation medium, forming a cannabinoid-comprising fermentation medium;
(iv) contacting the cannabinoid-comprising fermentation medium with the organic solvent, whereby secreted cannabinoid transfers into the organic solvent forming a cannabinoid-comprising organic solvent and a cannabinoid-depleted fermentation medium; and
(v) separating the cannabinoid-containing organic solvent from the cannabinoid-depleted fermentation medium, whereby a separated organic solvent comprising the cannabinoid is formed.

According to a further aspect of some embodiments of the present invention, there is provided a method for producing at least one cannabinoid, the method comprising:
(i) providing at least one cell capable of producing at least one cannabinoid and capable of secreting the at least one cannabinoid;
(ii) providing an anion exchanger;
(iii) growing the cell in a growth vessel in an aqueous fermentation medium comprising a carbon source, whereby the cell produces and secretes the at least one cannabinoid into the fermentation medium, forming a cannabinoid-comprising fermentation medium;
(iv) contacting the cannabinoid-comprising fermentation medium with the anion exchanger, whereby secreted cannabinoid binds onto the anion exchanger, forming a cannabinoid-carrying anion exchanger and a cannabinoid-depleted fermentation medium; and
(v) separating the cannabinoid-carrying anion exchanger from the cannabinoid-depleted fermentation medium, whereby a separated anion exchanger carrying the cannabinoid is formed.

As used herein, the term “interface” refers to a common boundary between two liquid phases, wherein the two phases may exist as two layers having an interface therebetween, or wherein one phase may be present as droplets or regions within the other phase, such that the interface is the outer surface of the droplet or region.

As used herein, the term “anion exchanger” refers to an agent carrying a positive charge capable of reversibly binding negatively charged counterions or an agent carrying a basic function, capable of reversibly binding acids.

According to some embodiments, the cell is a host cell selected from the group consisting of bacteria and yeast.

According to some embodiments, the cell is a Cannabis plant cell.

According to some embodiments, the at least one cannabinoid is selected from the group consisting of tetrahydrocannabinolic acid (THCA), tetrahydrocannabinol (THC), cannabidiolic acid (CBDA), cannabidiol (CBD), cannabigerolic acid (CBGA), cannabigerol (CBG), cannabichromenic acid (CBCA), cannabichromene (CBC), tetrahydrocannabivarinic acid (THCVA), tetrahydrocannabivarin (THCV), cannabidivarinic acid (CBDVA), cannabidivarin (CBDV) and combinations thereof. According to a preferred embodiment, the at least one cannabinoid is CBGA and/or CBG.

According to some embodiments, the at least one cannabinoid comprises one, two, three, four or more than four cannabinoids.

According to some embodiments, the cell further produces and secretes at least one terpene, such as one, two, three, four, five, six, seven, eight or more than eight terpenes.

According to some embodiments, the solvent has solubility in water of less than 10% wt at ambient temperature, less than 8% wt, less than 6% wt, less than 4% wt or less than 2% wt.

According to some embodiments, the solvent is a vegetable oil. According to some such embodiments, the vegetable oil is selected from the group consisting of hemp oil, coconut oil, corn oil, canola oil, cottonseed oil, palm oil, peanut oil, canola oil, safflower oil, avocado oil, mustard oil, peanut oil, sesame oil, soybean oil, sunflower oil, olive oil and combinations thereof.

According to some embodiments, the solvent is approved for use in food. According to some embodiments, the solvent is a pharmaceutical excipient.

According to some embodiments, the solvent comprises a hydrocarbon, such as a C4 to C10 hydrocarbon. According to some embodiments, the solvent comprises an alkanol, such as C6 (hexanol) to C16 (hexadecanol). According to some embodiments, the solvent comprises an amine selected from the group consisting of primary amines (RNH2), secondary amines (R2NH), tertiary amines (R3N) and quaternary amines (R4N⁺). According to related embodiments, the solubility in water of said amine is less than 5%. According to some embodiments, the solvent comprises an amine and at least one of a hydrocarbon and an alkanol.

According to some embodiments, the organic solvent has a boiling point of between about minus 20° C. and 250° C. at atmospheric pressure, such as about minus 20° C., about minus 10° C., about 0° C., about 10° C., about 20° C., about 50° C., about 100° C., about 150° C., about 200° C., or about 250° C.

According to some embodiments, the organic solvent comprises an amine.

According to some embodiments, separating the cannabinoid-containing organic solvent from the cannabinoid-depleted fermentation medium comprises centrifugation.

According to some embodiments, the method further comprises separating the at least one cannabinoid from the separated cannabinoid-containing organic solvent to form separated cannabinoid. According to some such embodiments, separating the at least one cannabinoid from the separated cannabinoid-containing organic solvent comprises a step selected from the group consisting of solvent extraction, distillation, crystallization and combinations thereof. According to some embodiments, separating the cannabinoid-containing organic solvent from the cannabinoid-depleted fermentation medium is conducted simultaneously with growing.

According to some embodiments, the fermentation medium and the organic solvent are provided in a single growth vessel, wherein a first phase and a second phase in physical contact through an interface are formed, wherein the first phase is the fermentation medium and the second phase is an organic phase comprising the organic solvent, wherein the cell is present in the fermentation medium.

According to some embodiments, the fermentation medium is provided in a growth vessel and the organic solvent is provided in a liquid-liquid contactor, the method further comprising removing a portion of the cannabinoid-comprising fermentation medium from the growth vessel and separating at least a fraction of the cells from the cannabinoid-comprising fermentation medium prior to contacting the cannabinoid-comprising fermentation medium with the organic solvent in the contactor. According to some embodiments, the contactor is selected from the group consisting of mixer-settlers, membrane contactors and counter-current column contactors. According to some embodiments, separating the cell from the cannabinoid-containing fermentation medium comprises filtration or centrifugation.

According to some embodiments, the method further comprises separating at least one cannabinoid from the separated anion exchanger carrying the cannabinoid to form separated cannabinoid.

According to some embodiments, separating at least one cannabinoid from the separated anion exchanger carrying the cannabinoid comprises elution with an aqueous solution of a base, such as sodium hydroxide, potassium hydroxide, ammonia, or combinations thereof.

According to some embodiments, the fermentation medium and the anion exchanger are provided in a single growth vessel.

According to some embodiments, the fermentation medium is provided in a growth vessel and the anion exchanger is provided in a contactor, the method further comprising removing a portion of the cannabinoid-comprising fermentation medium from the growth vessel and separating at least a fraction of the cells from the cannabinoid-comprising fermentation medium prior to contacting the cannabinoid-comprising fermentation medium with the anion exchanger in the contactor.

According to some embodiments, the contactor is cylindrical.

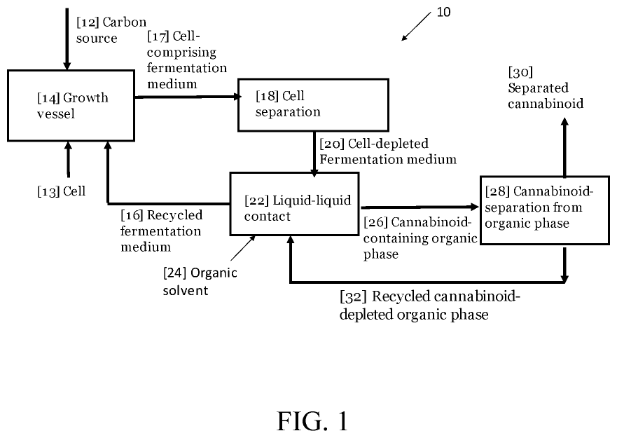

Referring now to FIG. 1, there is shown a schematic illustration of an exemplary embodiment of a method 10 in accordance with the principles of the present invention.

Method 10 comprises providing a cell 13 capable of producing and secreting at least one cannabinoid; providing a carbon source 12; providing a fermentation medium 16; growing cell 13 in a growth vessel 14 comprising fermentation medium 16 and carbon source 12 to form cannabinoid-comprising fermentation medium 17 comprising cells; separating 18 cell 13 from cell comprising fermentation medium 17 to form a cannabinoid-comprising fermentation medium 20 depleted of cells; contacting 22 cannabinoid comprising fermentation medium 20 with an organic solvent 24 to provide a liquid-liquid contact between fermentation medium 20 and organic solvent 24, forming cannabinoid-comprising organic solvent 26 and cannabinoid-depleted fermentation medium, recycled as 16; separating 28 cannabinoids from cannabinoid-comprising organic solvent 26 to provide separated cannabinoids 30 and cannabinoid-depleted organic solvent; and recycling 32 the cannabinoid-depleted organic solvent.

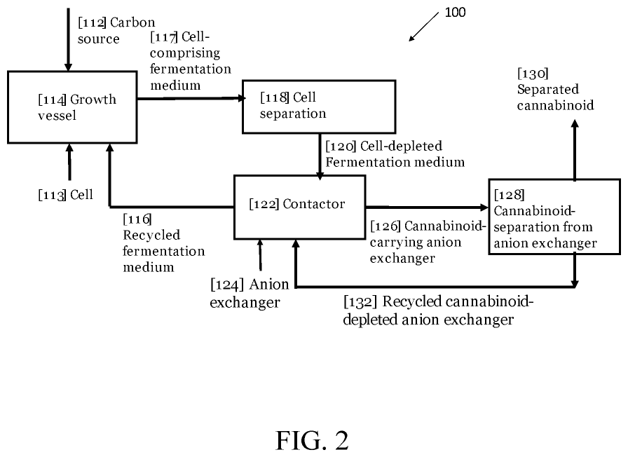

FIG. 2 provides a schematic illustration of an exemplary embodiment of a method 100 in accordance with the principles of the present invention.

Method 100 comprises providing a cell 113 capable of producing and secreting at least one cannabinoid; providing a carbon source 112; providing a fermentation medium 116; growing cell 113 in a growth vessel 114 comprising fermentation medium 116 and carbon source 112 to form cannabinoid-comprising fermentation medium 117 comprising cells; separating 118 cell 113 from cell comprising fermentation medium 117 to form a cannabinoid-comprising fermentation medium 120 depleted of cells; contacting 122 cannabinoid comprising fermentation medium 120 with a cannabinoid-carrying anion exchanger 124 whereby cannabinoid binds onto anion exchanger 124 forming a cannabinoid-carrying anion exchanger 126; and separating 128 the cannabinoid from cannabinoid-carrying anion exchanger 126, whereby a separated cannabinoid 130 and a cannabinoid-depleted anion exchanger 132 is formed; and optionally recycling cannabinoid-depleted anion exchanger 132 to contactor 122.

1. A method for producing at least one cannabinoid, the method comprising:
(i) providing at least one cell capable of producing at least one cannabinoid and capable of secreting said at least one cannabinoid;
(ii) providing an organic solvent;
(iii) growing said cell in a growth vessel in an aqueous fermentation medium comprising a carbon source, whereby said cell produces and secretes said at least one cannabinoid into said fermentation medium, forming a cannabinoid-comprising fermentation medium;
(iv) contacting said cannabinoid-comprising fermentation medium with said organic solvent, whereby secreted cannabinoid transfers into said organic solvent forming a cannabinoid-comprising organic solvent and a cannabinoid-depleted fermentation medium; and
(v) separating said cannabinoid-comprising organic solvent from said cannabinoid-depleted fermentation medium, whereby a separated organic solvent comprising said cannabinoid is formed.

2. A method for producing at least one cannabinoid, the method comprising:
(i) providing at least one cell capable of producing at least one cannabinoid and capable of secreting said at least one cannabinoid;
(ii) providing an anion exchanger;
(iii) growing said cell in a growth vessel in an aqueous fermentation medium comprising a carbon source, whereby said cell produces and secretes said at least one cannabinoid into said fermentation medium, forming a cannabinoid-comprising fermentation medium;
(iv) contacting said cannabinoid-comprising fermentation medium with said anion exchanger, whereby secreted cannabinoid binds onto said anion exchanger, forming a cannabinoid-carrying anion exchanger and a cannabinoid-depleted fermentation medium; and
(v) separating said cannabinoid-carrying anion exchanger from said cannabinoid-depleted fermentation medium, whereby a separated anion exchanger carrying said cannabinoid is formed.

3. The method of claim 1, wherein said cell is a host cell selected from the group consisting of bacteria and yeast.

4. The method of claim 1, wherein said cell is a Cannabis plant cell.

5. The method of claim 1, wherein said at least one cannabinoid is selected from the group consisting of tetrahydrocannabinolic acid (THCA), tetrahydrocannabinol (THC), cannabidiolic acid (CBDA), cannabidiol (CBD), cannabigerolic acid (CBGA), cannabigerol (CBG), cannabichromenic acid (CBCA), cannabichromene (CBC), tetrahydrocannabivarinic acid (THCVA), tetrahydrocannabivarin (THCV), cannabidivarinic acid (CBDVA), cannabidivarin (CBDV) and combinations thereof.

6. The method of claim 1, wherein said at least one cannabinoid is CBGA and/or CBG.

7. The method of claim 1, wherein said cell further produces and secretes at least one terpene.

8. The method of claim 1, wherein said organic solvent has a boiling point of between minus 20° C. and 250° C. at atmospheric pressure.

9. The method of claim 1, wherein said organic solvent comprises an amine.

10. The method of claim 1, wherein said separating said cannabinoid-comprising organic solvent from said cannabinoid-depleted fermentation medium comprises centrifugation.

11. The method of claim 1, further comprising separating at least one cannabinoid from said separated cannabinoid-comprising organic solvent to form separated cannabinoid.

12. The method of claim 11, wherein said separating at least one cannabinoid from said separated cannabinoid-comprising organic solvent comprises a step selected from the group consisting of solvent extraction, distillation, crystallization and combinations thereof.

13. The method of claim 1, wherein said separating said cannabinoid-comprising organic solvent from said cannabinoid-depleted fermentation medium is conducted simultaneously with said growing.

14. The method of claim 1, wherein said fermentation medium and said organic solvent are provided in a single growth vessel, wherein a first phase and a second phase in physical contact through an interface are formed, wherein said first phase is said fermentation medium and said second phase is an organic phase comprising said organic solvent, wherein said cell is present in said fermentation medium.

15. The method of claim 1, wherein said fermentation medium is provided in a growth vessel and said organic solvent is provided in a liquid-liquid contactor, the method further comprising removing a portion of said cannabinoid-comprising fermentation medium from said growth vessel and separating at least a fraction of said cells from said cannabinoid-comprising fermentation medium prior to said contacting said cannabinoid-comprising fermentation medium with said organic solvent in said contactor.

16. The method of claim 15, wherein said contactor is selected from the group consisting of mixer-settlers, membrane contactors and counter-current column contactors.

17. The method of claim 1, further comprising, subsequent to said contacting said cannabinoid-comprising fermentation medium with said organic solvent, recycling said cannabinoid-depleted fermentation medium to said growth vessel.

18. The method of claim 2, further comprising separating at least one cannabinoid from said separated anion exchanger carrying said cannabinoid to form separated cannabinoid.

19. (canceled)

20. The method of claim 2, wherein said fermentation medium and said anion exchanger are provided in a single growth vessel,

21. The method of claim 2, wherein said fermentation medium is provided in a growth vessel and said anion exchanger is provided in a contactor, the method further comprising removing a portion of said cannabinoid-comprising fermentation medium from said growth vessel and separating at least a fraction of said cells from said cannabinoid-comprising fermentation medium prior to said contacting said cannabinoid-comprising fermentation medium with said anion exchanger in said contactor.

22. (canceled)
